(19) United States
(12) Patent Application Publication
(10) Pub. No.: US 2025/0205190 A1
(43) Pub. Date: Jun. 26, 2025

(54) NUTRITIONAL COMPOSITION

(71) Applicants: N.V. NUTRICIA, Zoetermeer (NL); NUTRICIA EARLY LIFE NUTRITION (SHANGHAI) CO., LTD., Shanghai (CN)

(72) Inventors: Sebastian Tims, Utrecht (NL); Thomas Ludwig, Utrecht (NL); Guus Roeselers, Utrecht (NL); Jan Knol, Utrecht (NL); Gregg Ward, Utrecht (NL); Haofang Jin, Shanghai (CN)

(73) Assignees: N.V. NUTRICIA, Zoetermeer (NL); NUTRICIA EARLY LIFE NUTRITION (SHANGHAI) CO., LTD., Shanghai (CN)

(21) Appl. No.: 19/078,423

(22) Filed: Mar. 13, 2025

Related U.S. Application Data
(63) Continuation of application No. PCT/EP2023/075259, filed on Sep. 14, 2023.

(30) Foreign Application Priority Data
Sep. 14, 2022 (WO) PCT/CN2022/118832

(51) Int. Cl. A61K 31/23; A23L 33/00; A61K 31/702; A61K 35/00; A61K 35/745; A61K 35/747; A61K 38/17; A61K 38/40; A61P 1/14; A61P 31/04

(52) U.S. Cl. CPC A61K 31/23; A23L 33/40; A61K 31/702; A61K 35/745; A61K 35/747; A61K 38/1709; A61K 38/40; A61P 1/14; A61P 31/04; A61K 2035/115

(57) ABSTRACT

A nutritional composition comprising sn-2 palmitic acid and lactoferrin for use in improving the gut microbiota in a human subject.

### TECHNICAL FIELD OF THE INVENTION

The present invention is in the field of nutrition and relates to a nutritional composition for improving gut microbiota in a human subject, said nutritional composition comprises a lipid component rich in sn-2 palmitic acid and lactoferrin.

### BACKGROUND OF THE INVENTION

The gastrointestinal microbiota is in close and continuous contact with epithelial and immune cells in the gut. This constant stimulation is essential for the development and functioning of the immune system.

The human gut microbiota is established during the first years of life and forms a symbiotic relationship with the host. A healthy gut microbiota early in life has been associated with optimal cognitive development of infants and reduced chances of developing diseases later in life. The bacterial colonization of the gastrointestinal tract of a new-born is therefore important for their development.

The formation of the microbiota in a child's digestive tract is shaped by many factors, the most important of which are the type of delivery, feeding and environmental factors. It is known that children born by C-section develop a more adult-like microbiota and experience a delay in developing a balanced microbiota with abundance of beneficial species. The microbiota of breast-fed babies is known to become typically dominated by Bifidobacterium, whereas the microbiota of formula-fed babies tends to be more diverse with higher numbers of Enterobacter, Enterococcus, Bacteroides and Clostridium.

In adults, an unhealthy gut microbiota has been associated with several diseases/conditions including chronic gastrointestinal disease, sleep disturbance or constant fatigue, skin irritation, and autoimmune conditions. A healthy gut microbiota not only prevents and/or treats the aforementioned conditions, but it was also found to improve longevity, healthy aging and mental health.

Recent studies have found that high levels of Proteobacteria are an indicator of microbiota dysbiosis. The term “Proteobacteria” as used herein refers to a major group (phylum) of bacteria and one of the most abundant phyla in the human gut microbiota. A common trait of Proteobacteria is the Gram negative staining. Based on phylogenetic analysis of 16S rRNA gene the Proteobacteria phylum is divided 6 into classes: Alphaproteobacteria, Betaproteobacteria, Gammaproteobacteria, Deltaproteobacteria, Epsilonproteobacteria, and Zetaproteobacteria. Many common human pathogens are found in the Proteobacteria phylum: for example, the Brucella and Rickettsia genera belonging to the Alphaproteobacteria class, Bordetella and Neisseria to the Betaproteobacteria class, Escherichia, Shigella, Salmonella, and Yersinia to the Gammaproteobacteria class, and Helicobacter to the Epsilobacteria class.

High levels of Proteobacteria have been associated with conditions sustained by various degrees of inflammation such as metabolic disorders and inflammatory bowel disease, as well as lung diseases, asthma, chronic obstructive pulmonary disease.

It is known in the art that some nutritional ingredients, besides prebiotic non-digestible oligosaccharides, beneficially affect the gut microbiota.

WO2012/091946 describes a method for inhibiting the adhesion of at least one pathogen in the gastrointestinal tract of a human comprising administering to the human a nutritional composition comprising: a) a fat or lipid source; b) a protein source; c) lactoferrin produced by a non-human source; and d) a prebiotic composition comprising a compound selected from the group consisting of galacto-oligosaccharide, polydextrose, and combinations thereof.

WO2009/082216 describes a composition comprising sphingophospholipid or its degradation product and at least one non-digestible carbohydrate for providing and/or maintaining an optimal intestinal microbiota.

Yaron (2013), doi: 10.1097/MPG.0b013e31827e1ee2., describes a study on the effect of β-palmitate content in infant formula on the intestinal microbiota of term infants.

Guo (2022), doi: 10.1039/d1fo03692k, describes a randomized, double-blind, parallel, controlled study on the effect of an infant formula containing sn-2 palmitate on fecal microbiota and metabolome profiles of healthy term infants.

Nevertheless, there is still a relevant need in the art for nutritional compositions which reduce the chance for opportunistic pathogens, while fostering an increase in beneficial bacteria and thereby improving gut microbiota and gut health.

### SUMMARY OF THE INVENTION

The inventors have surprisingly found that a nutritional composition comprising a combination of sn-2 palmitic acid and lactoferrin synergistically reduces the relative abundance of disadvantageous Proteobacteria, in particular Enterobacteriaceae, in the gut, while increasing the relative abundance of beneficial bacterial families, thereby improving the gut microbiota.

Without wishing to be bound by a theory, due to the reduction of Proteobacteria there is opportunity for beneficial bacteria families, such as Bacteroidaceae and/or Bifidobacteriaceae, to increase in relative abundance, providing an overall improvement of the gut microbiota.

Hence, a first aspect of the invention pertains a nutritional composition comprising:
a. 10-50 wt. % lipid component, by dry weight, said lipid component containing at least 5 wt. % palmitic acid based on total fatty acids and at least 15 wt. % of palmitic acid, based on total palmitic acid, is located at the sn-2 position of a triglyceride; and
b. 0.05-5 wt. % lactoferrin by weight of total protein;
   for use in improving the gut microbiota in a human subject, wherein the improvement of gut microbiota encompasses reducing Proteobacteria.

A second aspect of the invention pertains to a nutritional composition comprising:
a. 10-50 wt. % lipid component by dry weight of the composition, wherein the lipid component contains at least 5 wt. % palmitic acid based on total fatty acids and at least 15 wt. % of the palmitic acid, based on total palmitic acid, is in the sn-2 position of a triglyceride;
b. 5-20 wt. % protein by dry weight of the composition, from which:
   i. 0.05-5 wt. % is lactoferrin, preferably 0.4-2 wt. % is lactoferrin;
   ii. 4-40 wt. % is beta-casein, preferably 8-30 wt. % is beta-casein, wherein at least 75% by weight of the beta-casein is a beta-casein variant that has a proline at position 67 of the beta-casein amino acid sequence;
c. 20-80 wt. % digestible carbohydrates by dry weight of the composition;
d. 2-12 wt. % non-digestible carbohydrates by dry weight of the composition, preferably galacto-oligosaccharides (GOS) and fructo-oligosaccharides (FOS); and
e. 10⁴-10¹² cfu probiotic bacteria per 100 g in dry weight, preferably wherein the probiotic bacteria is selected from Bifidobacterium and/or Lactobacillus.

In one embodiment of the invention the human subject is an infant. Bottle-fed infants have a different gut microbiota compared to the gut microbiota of a breast-fed infant. It is considered beneficial to obtain a gut microbiota similar to the gut microbiota of breast-fed infants. For infants in the first few months after birth it is particularly advantageous to have an increase in Bifidobacteriaceae, because the gut microbiota of breast-fed infants is dominated by Bifidobacteriaceae. When infants start eating solid foods is becomes advantageous to have an increase in Bacteroidaceae, as this rise is also observed in breast-fed infants.

In another embodiment of the invention the human subject in an adult. For adults an increase in Bacteroidaceae and/or Bifidobacteriaceae is also considered beneficial, because these have been reported to be associated with the improvement of a number of human health conditions ranging from intestinal disorders, improved immune function, improved gut barrier function, to parameters of metabolic health and even nervous system related disorders.

### DETAILED DESCRIPTION OF THE INVENTION

The invention thus concerns a nutritional composition comprising:
a. 10-50 wt. % lipid component, by dry weight, said lipid component containing at least 5 wt. % palmitic acid based on total fatty acids and at least 15 wt. % of palmitic acid, based on total palmitic acid, is located at the sn-2 position of a triglyceride; and
b. 0.05-5 wt. % lactoferrin by weight of total protein;
   for use in improving the gut microbiota in a human subject, wherein the improvement of gut microbiota encompasses reducing Proteobacteria.

For some jurisdictions, the invention may also be worded as a method for improving the gut microbiota in a human subject, said method comprising the administration of a nutritional composition comprising
a. 10-50 wt. % lipid component, by dry weight, said lipid component containing at least 5 wt. % palmitic acid based on total fatty acids and at least 15 wt. % of palmitic acid, based on total palmitic acid, is located at the sn-2 position of a triglyceride; and
b. 0.05-5 wt. % lactoferrin by weight of total protein;
   wherein the improvement of gut microbiota encompasses reducing Proteobacteria.

For some jurisdictions, the invention may also be worded as the use of palmitic acid and lactoferrin in the manufacture of a nutritional composition for improving the gut microbiota in a human subject, wherein the improvement of gut microbiota encompasses reducing Proteobacteria and wherein the nutritional composition comprises
a. 10-50 wt. % lipid component, by dry weight, said lipid component containing at least 5 wt. % palmitic acid based on total fatty acids and at least 15 wt. % of palmitic acid, based on total palmitic acid, is located at the sn-2 position of a triglyceride; and
b. 0.05-5 wt. % lactoferrin by weight of total protein.

For some jurisdictions, ‘improvement of gut microbiota’ may be considered non-therapeutic. By definition, the words “non-therapeutic” exclude any therapeutic effect. Thus, for some jurisdictions, the invention may be worded as a non-therapeutic method of improving gut microbiota in a human subject, said method comprising administering to said human subject a nutritional composition comprising
a. 10-50 wt. % lipid component, by dry weight, said lipid component containing at least 5 wt. % palmitic acid based on total fatty acids and at least 15 wt. % of palmitic acid, based on total palmitic acid, is located at the sn-2 position of a triglyceride; and
b. 0.05-5 wt. % lactoferrin by weight of total protein;
   wherein the improvement of gut microbiota encompasses reducing Proteobacteria.

In the context of the invention, the following definitions are provided.

The term “lipid” or “lipid component” as used herein comprises one or more selected from the group consisting of triglycerides, free fatty acids, monoglycerides and diglycerides and polar lipids (such as phospholipids, cholesterol, glycolipids, sphingomyelin). The terms ‘lipid’, ‘fat’ and ‘oil’ are used interchangeably herein and have equal meaning.

The term “lactoferrin” as used herein, also known as lactotransferrin (LTF), refers to a multifunctional protein of the transferrin family. Lactoferrin is a globular glycoprotein with a molecular mass of about 80 kDa that is widely represented in various secretory fluids, such as milk, saliva, tears, and nasal secretions.

The term “microbiota” as used herein refers to the range of microorganisms that may be commensal, symbiotic, or pathogenic found in and on all multicellular organisms.

The term “gut microbiota” as used herein, represents the entire microbial community, including bacteria and archaea, that live in the digestive tract of a human subject.

Preferably, the Proteobacteria are Gammaproteobacteria, more preferably the Proteobacteria are Enterobacteriaceae and most preferably the Proteobacteria are one or more of the genus Bilophila, Citrobacter, Escherichia, Shigella, Enterococcus, Salmonella, Proteus, Morganella, Providencia, Klebsiella and Yersinia.

The term “improving gut microbiota in a human subject” as used herein refers to a decrease in the relative abundance of Proteobacteria in the gut microbiota of a human subject administered a nutritional composition according to the invention compared to the relative abundance of Proteobacteria in the gut microbiota of said human prior to starting administration of the nutritional composition according to the invention or compared to a human subject of similar age and health status who consumed a similar nutritional composition with:
i. 10-50 wt. % lipid component, by dry weight, said lipid component containing at least 5 wt. % palmitic acid based on total fatty acids and less than 15 wt. % of palmitic acid, based on total palmitic acid, is located at the sn-2 position of a triglyceride; and
ii. less than 0.05 wt. % lactoferrin by weight of total protein.

Preferably, the improvement of gut microbiota is by a ≥5% reduction of the relative abundance of Proteobacteria, preferably ≥20, more preferably ≥25% reduction of the relative abundance of Proteobacteria.

In a preferred embodiment, the improvement of gut microbiota is by reducing the relative abundance ratio of Proteobacteria with respect to Bifidobacteriaceae, preferably to a ratio under 2.5, more preferably under 2.3, even more preferably under 2.1.

Preferably, the human subject is at risk of having a compromised microbiota. Preferably, the human subject is at risk of having a compromised microbiota due to receiving or having received antibiotics and/or proton pump inhibitors.

In one embodiment, the human subject is preferably an adult.

In another embodiment, the human subject is preferably an infant. More preferably, the human subject is an infant aged 0-36 months, even more preferably 0-12 months, and most preferably 0-6 months.

If the human subject is an infant, preferably the infant is at risk of having a compromised microbiota and is selected from the group of infants born via caesarean section, preterm infants, infants born from a mother who received intrapartum antibiotics, infants receiving or having received antibiotics, infants receiving or having received proton pump inhibitors, formula fed infants, or combinations thereof.

If the human subject is an infant, the nutritional composition is preferably selected from infant formula, follow-on formula and young child formula. The terms “infant formula” or “follow-on formula” or “young child formula” as used herein mean that the composition is artificially made or in other words that it is synthetic. This means that the nutritional composition preferably is not unprocessed mammalian milk.

In the present invention, infant formula refers to nutritional compositions, artificially made, intended for infants of 0 to about 4 to 6 months of age and are intended as a substitute for human milk. Typically, infant formulae are suitable to be used as sole source of nutrition. Such infant formulae are also known as starter formula. Follow-on formula for infants starting with at 4 to 6 months of life to 12 months of life are intended to be supplementary feedings for infants that start weaning on other foods. Infant formulae and follow-on formulae are subject to strict regulations, for example for the EU regulations no. 609/2013 and no. 2016/127. In the present context, young child formula refers to nutritional compositions, artificially made, intended for infants of 12 months to 36 months, which are intended to be supplementary feedings for infants. In the context of the present invention, young child formula can also be named growing-up milk.

If the human subject is an infant, the nutritional composition is preferably an infant formula or a follow-on formula. More preferably the nutritional composition is an infant formula.

### Lipid Components

The nutritional composition comprises 10-50 wt. % lipid component by dry weight of the composition.

Preferably, the nutritional composition comprises 12-45 wt. % of lipid component by dry weight of the composition, more preferably 14-40 wt. % of lipid component by dry weight of the composition, most preferably 16-35 wt. % of lipid component by dry weight of the composition.

The lipid component provides preferably 30 to 60% of the total calories of the nutritional composition. More preferably the lipid component provides 35 to 55% of the total calories, most preferably the lipid component provides 40 to 50% of the total calories.

The lipid component is preferably present in an amount of 3 to 7 g per 100 kcal, more preferably in an amount of 4 to 6 g per 100 kcal and most preferably in an amount of 4.5 to 5.5 g per 100 kcal.

When in liquid form, e.g. as a ready-to-feed liquid, the nutritional composition preferably comprises 2.0 to 6.5 g lipid component per 100 ml, more preferably 3.0 to 4.0 g lipid component per 100 ml.

Preferred lipid sources are one or more of mammalian milk fat, vegetable oil, microbial oil, algal oil, fungal oil, egg lipid and fish oil. More preferred lipid sources are one or more of mammalian milk fat, vegetable oil, microbial oil and fish oil.

Preferred vegetable oil sources are linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, palm oil and palm kernel oil.

Preferably the composition comprises at least 70 wt. %, more preferably at least 80 wt. %, most preferably at least 90 wt. % triglycerides based on the lipid component.

Triglycerides comprise a glycerol moiety to which, via ester bonds, three fatty acid residues are attached, which may be the same or different, and which are generally chosen from saturated and unsaturated fatty acids containing 4 to 26 carbon atoms. Such triglycerides may differ in the fatty acid residues that are present and/or may differ in the respective position(s) of the fatty acid residues to the glycerol backbone (e.g. in the sn-1, sn-2 and/or sn-3 position).

### Sn-2 Palmitic Acid

Palmitic acid (PA), as used herein refers to palmitic acid and/or acyl chains (C16:0). In the context of the invention ‘wt. % based on total fatty acids’ refers to the wt. % based on the sum of the weight of both fatty acids and acyl chains.

The lipid component comprises at least 5 wt. % palmitic acid (PA) based on total fatty acids, preferably at least 6 wt. % PA based on total fatty acids, more preferably 7 to 50 wt. % PA based on total fatty acids and most preferably 8 to 40 wt. % PA based on total fatty acids.

In the lipid component, at least 15 wt. % of PA, based on total PA, is located at the sn-2 position of a triglyceride. Preferably, in the lipid component, at least 20 wt. % of the PA, more preferably 25 to 60 wt. % of the PA, and most preferably 30 to 55 wt. % of the PA, based on total PA, is in the sn-2 position of a triglyceride.

Most preferably, the lipid component contains 8 to 40 wt. % palmitic acid based on total fatty acids and 30 to 55 wt. % of the palmitic acid, based on total palmitic acid, is in the sn-2 position of a triglyceride.

Preferred lipid sources for increasing the level of sn-2 palmitic acid in the lipid component are mammalian milk fat and/or structured vegetable lipids which are rich in 1,3-dioleoyl-2-palmitoylglycerol (OPO). Most preferably the lipid source for increasing the level of sn-2 palmitic acid in the lipid component is mammalian milk fat.

The lipid component preferably comprises 1-60 wt. % mammalian milk fat by weight of the lipid component, more preferably 2-40 wt. % mammalian milk fat and most preferably 3-25 wt. % mammalian milk fat by weight of the lipid component.

Preferably the mammalian milk fat is a non-human mammalian milk fat. More preferably the mammalian milk fat is a derived from bovine, goat, donkey, sheep, buffalo, or camel milk and combinations thereof. More preferably the mammalian milk fat is derived from bovine, goat or sheep milk and combinations thereof. Most preferably the mammalian milk fat is bovine milk fat.

Preferably, the mammalian milk fat is used in the form of cream, anhydrous milk fat, butter oil, butter fat or butter.

Structured vegetable lipids are commercially available—e.g. from Loders Croklaan under the name Betapol™ and/or can be prepared in a manner known per se, for instance as described in EP 0698078 and/or EP 0758846. Another suitable source is InFat™ of Enzymotec.

Preferably the lipid component comprises 20-60 wt. % structured vegetable lipids by weight of the lipid component, more preferably 25-55 wt. % structured vegetable lipids and most preferably 30-50 wt. % structured vegetable lipids by weight of the lipid component.

In a preferred embodiment, the lipid component comprises 5-50 wt. % 1,3-dioleoyl-2-palmitoylglycerol (OPO) by weight of the lipid component, more preferably 10-40 wt. % OPO and most preferably 15-35 wt. % OPO by weight of the lipid component.

### Fatty Acid Composition

PUFA refers to polyunsaturated fatty acids and/or acyl chains with 2 or more unsaturated bonds; LC-PUFA refers to long chain polyunsaturated fatty acids and/or acyl chains comprising at least 20 carbon atoms in the fatty acyl chain and with 2 or more unsaturated bonds. n3 or omega-3 PUFA refers to polyunsaturated fatty acids and/or acyl chains with 2 or more unsaturated bonds with an unsaturated bond at the third carbon atom from the methyl end of the fatty acyl chain, n6 or omega-6 PUFA refers to polyunsaturated fatty acids and/or acyl chains with 2 or more unsaturated bonds with an unsaturated bond at the sixth carbon atom from the methyl end of the fatty acyl chain.

DHA refers to docosahexaenoic acid and/or acyl chain (22:6, n3); DPA refers to docosapentaenoic acid and/or acyl chain (22:5 n3). EPA refers to eicosapentaenoic acid and/or acyl chain (20:5 n3); ARA refers to arachidonic acid and/or acyl chain (20:4 n6). LA refers to linoleic acid and/or acyl chain (18:2 n6); ALA refers to alpha-linolenic acid and/or acyl chain (18:3 n3).

Preferably, the nutritional composition comprises PUFAs, preferably n-3 and/or n-6 PUFAs.

Preferably, the nutritional composition comprises n3 LC-PUFA, such as EPA, ALA and/or DHA, more preferably DHA. As the conversion of ALA to DHA may be less efficient in infants, preferably both ALA and DHA are present in the nutritional composition for infants.

Preferably the nutritional composition comprises 0.05-4 wt. %, more preferably 0.1-2 wt. %, and most preferably 0.2-1 wt. %, of DHA based on total fatty acids.

Preferably the nutritional composition comprises 0.1-4 wt. %, more preferably 0.2-2 wt. %, most preferably 0.4-1 wt. % of ARA based on total fatty acids.

Preferably the weight ratio between DHA and ARA is between 5:1 to 1:5, more preferably between 3:1 to 1:3, most preferably between 2:1 and 1:2.

### Protein

The nutritional composition preferably comprises protein. The term ‘protein’ as used herein refers to the sum of proteins, peptides and free amino acids.

Preferably, the nutritional composition comprises 5-20 wt. % protein, based on dry weight of the composition, more preferably 7-17 wt. % protein, most preferably 9-14 wt. % protein, based on dry weight of the composition.

Expressed differently, protein preferably provides 5 to 20% of the total calories of the nutritional composition, preferably 6 to 12% of the total calories of the nutritional composition.

Preferably, the nutritional composition comprises 1.5 to 4 g protein per 100 kcal, more preferably 1.7 to 3 g protein per 100 kcal.

Based on a ready-to-drink liquid product, protein preferably provides 0.6 to 3 g per 100 ml, more preferably 0.8 to 2.5 g per 100 ml, and most preferably between 1 and 2 g per 100 ml.

Preferred protein sources are dairy protein, vegetable protein, or mixtures thereof. Examples of suitable vegetable protein are soy protein, pea protein, potato protein and combinations thereof.

Preferred dairy protein sources are a derived from bovine, goat, donkey, sheep, buffalo, or camel milk and combinations thereof. Preferably the dairy protein is bovine dairy protein.

The dairy protein preferably comprises whey protein, casein and combinations thereof. The nutritional composition preferably comprises casein and whey proteins in a weight ratio casein: whey protein of 10:90 to 90:10, more preferably 20:80 to 80:20.

### Lactoferrin

Preferably, the nutritional composition comprises 0.1-4 wt. % of lactoferrin by weight of total protein, more preferably 0.2-3 wt. % of lactoferrin by weight of total protein, and most preferably 0.4-2 wt. % of lactoferrin by weight of total protein.

In one embodiment, the lactoferrin is non-denatured lactoferrin. The term “non-denatured lactoferrin” as used herein refers to lactoferrin molecules whose tertiary structure is preserved. Non-denatured lactoferrin is therefor still bioactive. Preferably, at least 80 wt. %, more preferably at least 90 wt. %, most preferably at least 95 wt. % of the total lactoferrin in the nutritional composition is non-denatured lactoferrin.

Preferably the lactoferrin is derived from bovine, goat, donkey, sheep, buffalo, or camel milk and combinations thereof. More preferably, the lactoferrin is bovine lactoferrin.

### Beta-Casein

Beta-caseins can be categorized as A1 beta-casein and A2 beta-casein. These two proteins are the predominant beta-caseins in bovine milk consumed by most human populations. A1 beta-casein differs from A2 beta-casein by a single amino acid. A histidine amino acid is located at position 67 of the 209 amino acid sequence of A1 beta-casein, whereas a proline is located at the same position of A2 beta-casein. This single amino acid difference is, however, critically important to the enzymatic digestion of beta-caseins in the gut. The A1 beta-casein type is the most common type found in bovine's milk in Europe and in the United States.

As used herein, a beta-casein variant that has a proline at position 67 of the beta-casein amino acid sequence preferably refers to A2 beta-casein. However, one skilled in the art will appreciate that it may be any A2 type beta-casein variant, i.e. including any but not limited to A2, A3, D and E beta-caseins which have proline at the homologous position of the beta-casein amino acid sequence.

The nutritional composition preferably comprises beta-casein, wherein at least 75% by weight of the beta-casein is a beta-casein variant that has a proline at position 67 of the beta-casein amino acid sequence.

Preferably, the nutritional composition comprises at least 80 wt. %, more preferably at least 90 wt. %, even more preferably at least 98 wt. % by weight of the beta-casein is a beta-casein variant that has a proline at position 67 of the beta-casein amino acid sequence. Most preferably, 100 wt. % of the beta-casein is a beta-casein variant that has a proline at position 67 of the beta-casein amino acid sequence.

Preferably the beta-casein is derived from bovine, goat, donkey, sheep, buffalo, or camel milk and combinations thereof. More preferably the beta-casein is bovine beta-casein.

Preferably, the nutritional composition comprises 4-40 wt. %, more preferably 6-35 wt. %, most preferably 8-30 wt. % of beta-casein by weight of total protein.

### Digestible Carbohydrates

The nutritional composition preferably comprises digestible carbohydrates. Based on dry weight the nutritional composition preferably comprises 20 to 80 wt. %, more preferably 40 to 65 wt. % digestible carbohydrates.

Preferably, digestible carbohydrates provide 25 to 75% of the total calories of the nutritional composition, more preferably the digestible carbohydrates provide 35 to 55% of the total calories.

Based on calories the nutritional composition preferably comprises of 5 to 20 g of digestible carbohydrates per 100 kcal, more preferably 6 to 16 g per 100 kcal.

When in liquid form, e.g. as a ready-to-feed liquid, the nutritional composition preferably comprises 3 to 20 g digestible carbohydrate per 100 ml, more preferably 4 to 15 g per 100 ml, even more preferably 5 to 10 g digestible carbohydrate per 100 ml.

Preferred digestible carbohydrate sources are lactose, glucose, sucrose, fructose, galactose, maltose, starch and maltodextrin. The nutritional composition preferably comprises lactose.

### Non-Digestible Oligosaccharides

The nutritional composition preferably comprises non-digestible oligosaccharides (NDO), preferably one or more NDO selected from galacto-oligosaccharides (GOS) and fructo-oligosaccharides (FOS).

The nutritional composition preferably comprises 2 to 12 wt. % NDO, more preferably 3 to 10 wt. % NDO, most preferably 4 to 8 wt. % NDO, based on dry weight of the nutritional composition.

Preferably GOS are transgalactooligosaccharides. Suitable non-digestible oligosaccharides are for example Vivinal@GOS (FrieslandCampina DOMO), Raftilin®HP or Raftilose® (Orafti).

### Probiotic

The nutritional composition preferably comprises probiotic bacteria. The probiotic bacteria content of the nutritional composition is preferably in the range 10⁴-10¹² cfu/100g of dry weight, more preferably 10⁷-10¹¹ cfu/100 g of dry weight.

Preferably the probiotic bacteria is selected from Bifidobacterium and/or Lactobacillus. More preferably, the nutritional composition comprises Bifidobacterium, even more preferably Bifidobacterium breve, and most preferably Bifidobacterium breve M-16V.

### Nutritional Composition

In a preferred embodiment, the nutritional composition is a powder. Preferably, the powder is reconstituted with aqueous liquid to prepare a liquid formula and this liquid formula is subsequently orally administered to the human subject. Preferably, the aqueous liquid is water.

In an alternative preferred embodiment, the nutritional composition is a concentrated liquid, a supplement, or a ready-to-drink nutritional composition.

The second aspect of the invention is a nutritional composition comprising:
a. 10-50 wt. % lipid component by dry weight of the composition, wherein the lipid component contains at least 5 wt. %, preferably 8 to 40 wt. %, palmitic acid based on total fatty acids and at least 15 wt. %, preferably 30-55 wt. %, of the palmitic acid, based on total palmitic acid, is in the sn-2 position of a triglyceride;
b. 5-20 wt. % protein by dry weight of the composition, from which:
   i. 0.05-5 wt. % is lactoferrin, preferably 0.4-2 wt. % is lactoferrin;
   ii. 4-40 wt. % is beta-casein, preferably 8-30 wt. % is beta-casein,
      wherein at least 75% by weight of the beta-casein is a beta-casein variant that has a proline at position 67 of the beta-casein amino acid sequence;
c. 20-80 wt. % digestible carbohydrates by dry weight of the composition;
d. 2-12 wt. % non-digestible carbohydrates by dry weight of the composition, preferably galacto-oligosaccharides (GOS) and fructo-oligosaccharides (FOS); and
e. 10⁴-10¹² cfu probiotic bacteria per 100 g in dry weight, preferably wherein the probiotic bacteria is selected from Bifidobacterium and/or Lactobacillus.

Preferably, the nutritional composition is for improving the gut microbiota in a human subject, wherein the improvement of gut microbiota encompasses reducing Proteobacteria. More preferably, the gut microbiota is improved by a ≥5%, preferably ≥20%, more preferably ≥25%, reduction of the relative abundance of Proteobacteria.

Preferably, all embodiments presented herein before, equally apply to the first and the second aspect of the invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Example 1

In an in vitro colon microbiota model, i-screen™ (TNO, Zeist, Netherlands), the effect of infant milk formula ingredients on the anaerobic fermentation of infant microbiota was assessed.

### Tested Ingredients

The in vitro microbiota model mimics colonic conditions. Before a consumed ingredient reaches the colon in a human, already a part of the digestion/absorption has taken place in the gastrointestinal tract preceding the colon. For each ingredient the degree of digestion/absorption before reaching the colon varies and this was taken into account when selecting the ingredient concentrations to be tested (see Table 1).

Lactoferrin is a glycosylated protein and has been described to be resistant to digestion in vitro and in vivo. Fat is not completely digested by infants, therefor approximately 1.5% of the fat concentration in the formula were used. GOS and FOS are a prebiotic fiber, which is neither digested nor absorbed in the gastrointestinal tract before reaching the colon.

TABLE 1

| | | Ingredient | | |
|---|---|---|---|---|
| | Ingredient | conc. in well | | Source of ingredient |
| A | Lactoferrin | 2 | mg/ml | Friesland Campina, Vivinal |
| | | | | Lactoferrin Comprises |
| | | | | 96 wt. % lactoferrin. |
| B | Milk fat | 21.4 | μg/ml | Friesland Campina, Anhydrous |
| | | | | milk fat (see Table 2) |
| C | scGOS/lcFOS | 2 | mg/ml | scGOS to lcFOS ratio = 9:1 |

| | | | |
|---|---|---|---|
| | | | Friesland Campina, Vivinal GOS, |
| | | | 45% w/v scGOS |
| | | | Raftiline HP, >0.95 g/g lcFOS |
| 1 | Lactoferrin | 2 mg/ml | Same as for A & B |
| | Milk fat | 21.4 μg/ml | |
| 2 | Lactoferrin | 2 mg/ml | Same as for A & B & C |
| | Milk fat | 21.4 μg/ml | |
| | scGOS/lcFOS | 2 mg/ml | |

TABLE 2

| | Anhydrous |
|---|---|
| | milk fat |
| Palmitic acid (wt. % by weight of fatty acids) | 30.6 |
| % Palmitic acid of total palmitic acid in sn-2 position | 45% |

### Microbiota Sample

The infant faecal pool consisted of samples obtained from faeces of 5 European infants aged 3.5-8.5 months. The samples were thawed, homogenized, aliquoted and stored at −80° C. till the start of the i-screen assay run.

### i-Screen Assay

Before starting the fermentation with the test ingredients. The infant fecal pool was incubated in an anaerobic flask for 4 hours in SIEM (simulated ileal environment media) medium (pH 5.8) under anaerobic conditions (37° C.; 300 rpm). Subsequently this SIEM medium with feces was homogenized and distributed into a 96 w-plate.
The test ingredients were pre-diluted in a higher concentration in fresh SIEM medium and added to the existing medium in the 96 wells plate. For milk fat the addition method was slightly adapted, milk fat was added to fresh SIEM medium at 45° C., subsequently thoroughly homogenized, followed by addition to the wells in the 96 w plate.
The i-screen experiment included an untreated control (microbiota only), an positive control (Inulin, Raftiline ST), blanks (SIEM medium only) and technical controls (internal controls for DNA isolation and sequencing).
Each test condition was conducted in triplicate.
After 24 hours of fermentation in SIEM medium under anaerobic conditions at 37° C., samples were collected for DNA analyses.

### 16s rRNA Gene Amplicon Sequencing

Total DNA from collected samples was isolated using the DNeasy 96 Powersoil Pro QIAcube HT kit (47014, Qiagen) on a QIAcube with minor adjustments. To each 100 μl sample, 500 μl zirconium beads (0.1 mm) and 800 μl CD1 solution (DNeasy 96 Powersoil Pro QIAcube HT kit) were added. Cells were disrupted by bead beating (2×2 min with cooling on ice in between and afterwards). After centrifugation (6 min 3000 RPM), 600 μl supernatant was mixed with 300 μl CD2 solution (DNeasy 96 Powersoil Pro QIAcube HT kit) and followed by another centrifugation step (6 min 3000 RPM). Of the supernatant, 550 μl was then used for further extraction using the QIAcube according to the manufacturer's instructions.

Changes in the microbiota composition were analyzed by using 16S rDNA amplicon sequencing. The V4 hypervariable region was targeted. 100 pg of DNA was amplified as described by Kozich et al. [https://doi.org/10.1128/AEM.01043-13] with the exception that 30 cycles were used instead of 35, applying F515/R806 primers [doi: 10.1073/pnas. 1000080107]. The amplicon libraries were pooled in equimolar amounts and purified using the QIAquick Gel Extraction Kit (QIAGEN). Amplicon quality and size were analyzed on a Fragment Analyzer (Advanced Analytical Technologies, Inc.). Paired-end sequencing of amplicons (approximately 400 base pairs) was conducted on the Illumina MiSeq platform (Illumina, Eindhoven, The Netherlands).

Sequence pre-processing, analysis, and classification was performed using modules implemented in the Mothur software platform [https://doi.org/10.1128/AEM.01541-09]. Chimeric sequences were identified and removed using the chimera.uchime command. 16S rDNA unique sequences were aligned using the ‘align.seqs’ command and the Mothur-compatible Bacterial SILVA SEED database (Release 119). Taxonomic classification was performed using the RDP-II Naïve Bayesian Classifier using a 60% confidence threshold against the RDP Database (Release 11.1) for 16S rRNA. Taxonomic classification was performed at the genus level.

### Analysis of Data

The annotated sequence data were pooled per taxonomic group and the relative abundance of each taxonomic group was determined per sample.

TABLE 3
Results at t = 24 hours

| | Average relative abundance (%)—[st. dev] | | |
|---|---|---|---|
| | Enterobacteriaceae | Bacteroidaceae | Bifidobacteriaceae |
| A | 41.73 (1.64) | 19.81 (0.48) | 9.39 (1.50) |
| B | 40.61 (1.1) | 17.95 (0.38) | 11.88 (1.09) |
| C | 45.84 (1.70) | 3.94 (0.10) | 22.93 (2.84) |
| 1 | 26.32 (0.21) | 19.78 (0.90) | 12.77 (0.37) |
| 2 | 32.97 (0.54) | 2.97 (0.36) | 19.27 (0.28) |

Before incubation with the test ingredients started, the relative abundance of the Enterobacteriacea was 49.89%, 2.21% [st. dev]. All three single ingredients (A-C) reduced the relative abundance of the Enterobacteriaceae. Due to the reduction in Enterobacteriaceae, there was opportunity for beneficial bacteria families, such as Bacteroidaceae and/or Bifidobacteriaceae, to increase in relative abundance. When lactoferrin and milk fat were combined in experiment 1 the relative abundance of Enterobacteriaceae was synergistically reduced. The ratio of Enterobacteriaceae to Bifidobacteriaceae for experiment 1 was reduced compared to the three single ingredients (A-C). Similar results were observed when lactoferrin and milk fat were combined with GOS/FOS in experiment 2.

1. A method for improving gut microbiota in a human subject, the method comprising administrating to the subject a nutritional composition comprising:
a. 10-50 wt. % lipid component, by dry weight, said lipid component containing at least 5 wt. % palmitic acid based on total fatty acids and at least 15 wt. % of palmitic acid, based on total palmitic acid, is located at the sn-2 position of a triglyceride, wherein the composition comprises at least 70 wt. % triglycerides, based on the lipid component; and
b. 5-20 wt. % protein by dry weight, wherein 0.05-5 wt. % lactoferrin by weight of total protein;
wherein the method for improving gut microbiota encompasses reducing Proteobacteria.

2. The method according to claim 1, wherein the improvement of gut microbiota further encompasses increasing Bacteroides.

3. The method according to claim 1, wherein the lipid component contains 8 to 40 wt. % palmitic acid based on total fatty acids and 30 to 55 wt. % of the palmitic acid, based on total palmitic acid, is in the sn-2 position of a triglyceride.

4. The method according to claim 1, wherein the lipid component comprises at least one of:
(i) 1-60 wt. % mammalian milk fat by weight of the lipid component;
(ii) 20-60 wt. % structured vegetable lipids by weight of the lipid component; and
(iii) 5-50 wt. % OPO (1,3-dioleoyl-2-palmitoylglycerol) by weight of the lipid component.

5.-6. (canceled)

7. The method according to claim 1, wherein the nutritional composition further comprises non-digestible oligosaccharides (NDO).

8. The method according to claim 7, wherein the improvement of gut microbiota further encompasses increasing Bifidobacterium.

9. The method according claim 1, wherein the nutritional composition further comprises at least one of:
(i) beta-casein, wherein at least 75% by weight of the beta-casein is a beta-casein variant that has a proline at position 67 of the beta-casein amino acid sequence;
(ii) probiotic bacteria.

10. The method according to claim 7, wherein the probiotic bacteria comprise Bifidobacterium and/or Lactobacillus.

11. The method according to claim 1, wherein the gut microbiota is improved by a ≥5% reduction of the relative abundance of Proteobacteria.

12. The method according to claim 1, wherein the human subject is at risk of having a compromised microbiota.

13. The method according to claim 12, wherein the human subject is at risk of having a compromised microbiota due to receiving or having received antibiotics and/or proton pump inhibitors.

14. The method according to claim 1, wherein the human subject is an adult.

15. The method according to claim 1, wherein the human subject is an infant.

16. The method according to claim 15, wherein the nutritional composition is selected from infant formula, follow-on formula and young child formula.

17. The method according to claim 15, wherein the infant is at risk of having a compromised microbiota and is selected from the group of infants born via caesarean section, preterm infants, infants born from a mother who received intrapartum antibiotics, infants receiving or having received antibiotics, infants receiving or having received proton pump inhibitors, formula fed infants, or combinations thereof.

18.-20. (canceled)
